(19) United States
(12) Patent Application Publication
(10) Pub. No.: US 2025/0340821 A1
(43) Pub. Date: Nov. 6, 2025

(54) Bioreactor Systems

(71) Applicant: ABEC, Inc., Bethlehem, PA (US)

(72) Inventors: Christopher Boyce, Bethlehem, PA (US); Paul Kubera, Bethlehem, PA (US); Nicholas Leight, Bethlehem, PA (US); Daniel Miller, Bethlehem, PA (US); Eric Dyke, Bethlehem, PA (US)

(73) Assignee: ABEC, Inc., Bethlehem, PA (US)

(21) Appl. No.: 18/862,605

(22) PCT Filed: May 5, 2023
(86) PCT No.: PCT/US2023/021167

§ 371 Date: Nov. 4, 2024

Related U.S. Application Data
(60) Provisional application No. 63/339,124, filed on May 6, 2022.
(60) Provisional application No. 63/342,379, filed on May 16, 2022.

(51) Int. Cl. C12M 1/36; C12M 1/00; C12M 1/02; C12M 1/06

(52) U.S. Cl. CPC C12M 41/48; C12M 27/02; C12M 29/06; C12M 29/10; C12M 41/22; C12M 47/16

(57) ABSTRACT

This disclosure relates to large-scale bioreactor systems comprising a vessel comprising internal reaction chamber having a volumetric and cell-sustaining capacity significantly above that of currently available bioreactor systems.

### RELATED APPLICATIONS

This application is a 35 U.S.C. § 371 national stage application of International Application No. PCT/US2023/021167, filed on May 5, 2023, and claims priority to U.S. Ser. No. 63/339,124 filed on May 6, 2022 and U.S. Ser. No. 63/342,379 filed on May 16, 2022; each of which is incorporated into this application in its entirety for any purpose.

### FIELD OF THE DISCLOSURE

This disclosure relates to large-scale bioreactor systems comprising a vessel comprising an internal reaction chamber having a volumetric and cell-sustaining capacity significantly above that of currently available bioreactor systems.

### BACKGROUND OF THE DISCLOSURE

Currently available bioreactor systems are limited in both size (e.g., volume) and cell-sustaining capacity. For instance, those of ordinary skill in the art recognize that currently available bioreactors cannot support high volume of high-density non-bacterial cell cultures. This disclosure provides solutions to these problems. For instance, in some embodiments, this disclosure provides bioreactor systems comprising a vessel having an internal reaction chamber of at least about 125,000 L to 315,000 L and a viable cell density of, e.g., greater than 50 million cells per milliliter. This disclosure thereby providing solutions to these and other art-recognized, and unrecognized, problems.

### BRIEF DESCRIPTION OF THE DRAWINGS

FIG. 1. First exemplary bioreactor train.

FIG. 2. Second exemplary bioreactor train.

FIG. 3. First exemplary impeller.

FIG. 4. Second exemplary impeller.

FIG. 5. Third exemplary impeller.

FIG. 6. Fourth exemplary impeller.

FIG. 7A. Exemplary sparger design.

FIG. 7B. Exemplary sparger design showing openings through which gas traverses from a supply to the interior chamber of the bioreactor vessel.

FIG. 8. Exemplary bioreactor vessel and associated components (bioreactor system).

FIG. 9. Exemplary manufacturing process.

FIG. 10. Exemplary bioreactor system with automation.

FIG. 11. Exemplary bioreactor system with automation.

FIG. 12. Exemplary bioreactor system with automation.

### SUMMARY OF THE DISCLOSURE

This disclosure relates to large-scale bioreactor systems comprising a vessel (e.g., bioreactor vessel) comprising an internal reaction chamber having a volumetric and cell-sustaining capacity significantly above that of currently available bioreactors. In preferred embodiments, this disclosure provides Aspects 1-15 described below. In preferred embodiments, the bioreactor vessel comprises an internal reaction chamber configured to contain at least about 125,000 liters L, at least about 250,000 L, or at least about 315,000 L of a reaction mixture comprising cells, liquid, and gas. In preferred embodiments, the bioreactor vessel has a liquid depth (LD) to bioreactor vessel diameter (D) ratio of 1.5-3, the ratio optionally being about 2.4, optionally in preferred embodiments about 2.36. Other embodiments are also disclosed herein as would be understood by those of ordinary skill in the art.

### DETAILED DESCRIPTION OF THE INVENTION

This disclosure relates to bioreactor systems comprising at least one vessel (e.g., bioreactor vessel) comprising an internal reaction chamber having a reaction mixture capacity or volume of at least about 30,000 liters (L), at least about 50,000 L, at least about 75,000 L, at least about 100,000 L, at least about 125,000 liters L, at least about 250,000 L, or at least about 315,000 L, preferably at least about 125,000 L to 315,000 L, even more preferably at least about 250,000 L.

In some embodiments, the vessel can have a form and/or used in a system disclosed in, for instance and without limitation, U.S. Pat. No. 8,658,419 (ABEC, Inc.), U.S. Pat. No. 9,228,165 B2 (ABEC, Inc.), U.S. Pat. No. 10,519,415 B2, and/or WO 2019/070648 A2 In some embodiments, the disposable feed vessel can be a cone-bottom or tulip-bottom vessel and/or preferably has a capacity of at least 20 L. Other types of suitable feed vessels that could be used as disclosed herein are also known in the art as would be understood by those of ordinary skill in the art. The materials used to produce the equipment described herein may be of the same or different composition. The reactor vessels and/or heat exchange components described herein are typically but not necessarily constructed from a corrosion-resistant alloy (e.g., metal). For instance, suitable materials may include, without limitation, dimple-jacket material and/or sheet/plate stock. Suitable materials include, for example, carbon steel, stainless steel (e.g., 304, 304L, 316, 316L, 317, 317L, AL6XN), aluminum, Inconel® (e.g., Inconel 625, Chronin 625, Altemp 625, Haynes 625, Nickelvac 625 and Nicrofer 6020), Incoloy®, Hastelloy (e.g., A, B, B2, B3, B142T, Hybrid-BC1, C, C4, C22, C22HS, C2000, C263, C276, D, G, G2, G3, G30, G50, H9M, N, R235, S, W, X), and Monel®, titanium, Carpenter 20®, among others. It is understood, however, that other materials besides or in addition to a corrosion-resistant alloy such as, but without limitation, plastic, rubber, and mixtures of such materials may also be suitable. A “mixture” of materials may refer to either an actual mixture per se to form a combined material or the use of various materials within the system (e.g., an alloy reactor shell and rubber baffle components). Regarding the channeled material referred to above, any of the suitable materials described above may be prepared such that channels are formed through which heat transfer media may be distributed.

The reaction vessel comprises an internal chamber, and in preferred embodiments is associated with and/or includes at least heat transfer system comprising a heat transfer apparatus for controlling the temperature of a chemical, pharmaceutical or biological process being carried out in within an internal reaction chamber of the vessel. In some embodiments, the heat transfer system provides for distribution of a heat transfer medium such that heat resulting from or required by the process is transferred from or to the reaction mixture. In some embodiments, the reaction vessel comprises a jacket and/or a jacketed tank head that provides a fluidic channel through which a heat transfer fluid may be circulated (e.g., a dimple jacket). In some embodiments, the reaction vessel may be a least partially surrounded by a fluidic channel. The jacketed tank head may also act as a lid for the reaction vessel. The jacketed tank head may also serve to support and/or relieve pressure on a DC (e.g., on the top of the DC) contained within the reactor vessel.

In some embodiments, the one or more heat exchange systems may comprise jacket through which a heat transfer fluid is circulated. The jacket may, for instance, comprises channels through which the heat transfer fluid is circulated. In some embodiments, the jacket may be a “dimpled” material. Dimple jackets are typically installed around reaction vessels such as fermentation tanks and may be used as part of a heat transfer system. Dimple jacket material may be used in the devices described herein in the typical fashion, e.g., wrapped around the reaction vessel. In certain embodiments described herein, dimple jacket material may be also or alternatively used within the baffle structure. Dimple jacket materials are commercially available, and any of such materials may be suitable for use as disclosed here. Typically, dimple jacket materials have a substantially uniform pattern of dimples (e.g., depressions, indentations) pressed or formed into a parent material (e.g., a sheet of metal). Dimple jacket materials may be made mechanically (“mechanical dimple jacket”) or by inflation (e.g., inflated resistance spot welding (RSW)), for example. To prepare a mechanical dimple material, a sheet of metal having a substantially uniform array of dimples pressed into, where each dimple typically contains a center hole, is welded to the parent metal through the center hole. An inflated RSW dimple material (e.g., inflated HTS or H.T.S.) is typically made by resistance spot welding an array of spots on a thin sheet of metal to a more substantial (e.g., thicker) base material (e.g., metal). The edges of the combined material are sealed by welding and the interior is inflated under high pressure until the thin material forms a pattern of dimples. Mechanical dimple materials, when used as jackets, typically have high pressure ratings and low to moderate pressure drop, while RSW dimple jackets typically exhibit moderate pressure ratings and a high to moderate pressure drop. Heat transfer fluid typically flows between the sheets of dimpled material. Other suitable dimple materials are available to those of skill in the art and would be suitable for use as described herein.

In preferred embodiments, the reactor vessels disclosed herein comprise one or more heat transfer systems that efficiently transfer heat, withstand the hydraulic forces encountered within a reaction vessel, and may be simply and efficiently sanitized. A suitable heat transfer baffle described herein may be incorporated into heat transfer systems to solve these problems. Preferred exemplary heat transfer baffles are disclosed and/or claimed in U.S. Pat. No. 8,658,419 B2, which is incorporated herein in its entirety, such as that disclosed herein is illustrated in FIGS. 16-18. In certain embodiments, the baffle has at least one internal channel (e.g., 9 in FIGS. 16-18) and at least two external channels (e.g., 10 in FIGS. 16-18). Typically, heat transfer media is circulated through one or more distribution channels (e.g., 9 in FIGS. 16-18) but not the one or more relief channels (e.g., 10 in FIGS. 16-18), which may also function as a vent(s) for the distribution channels. Distribution channels 9 are typically formed between the support material 11 and dimple jacket material 12 of each sub-assembly. Relief channel(s) 10 are typically formed by adjoining two sub-assemblies, each comprising support material 11 fixably attached to dimple jacket material 12 to one another. In such embodiments, the dimple jacket material and support material of each sub-assembly are typically adjoined to one another by welding or other process resulting in the materials being fixably attached to one another. The sub-assemblies are typically adjoined to one another using closure bars 13. The closure bar is typically adjoined to the support material by a welding or other process that results in a substantially seamless joint. The width of the closure bar may be adjusted to set the width of the relief channel as desired (e.g., setting the juxtaposed dimple jacket material closer together or further apart). One or more relief holes may be made within the closure bars such that relief channel(s) may communicate with the reaction vessel exterior. The incorporation of distribution and relief channels into the baffle provides exceptional heat transfer capabilities and the structural integrity necessary to withstand the hydraulic forces encountered in a reaction vessel. The baffles may protrude at regular or irregular intervals from the inner wall of the reaction vessel. The baffles may also be installed at any suitable angle relative to the inner wall of the reaction vessel (e.g., 60° relative to the interior wall, 30° relative to the radius of the reactor vessel). A suitable angle may be an angle that would be understood by the skilled artisan to be appropriate in order to or sufficient to attenuate the forces (e.g., hydraulic forces) encountered by the baffles resulting from motion (e.g., rotational and/or swirl motion) of the vessel contents resulting from the agitation (e.g., mechanical or otherwise) thereof. A suitable angle is one that would prevent damage to the baffles from the forces resulting from such motion. Suitable angles include, for example, 5°, 10°, 15°, 20°, 25°, 30°, 35°, 40°, 45°, 50°, 55°, 60°, 65°, 70°, 75°, 80°, 85°, or 90° relative to either the interior wall of the vessel or the radius of the vessel. Where the reaction vessel contains a mechanism (e.g., mechanical or other mechanism) for agitating or mixing a reaction, such as a set of rotating blades or the like (e.g., an axial flow or radial flow impeller), the baffles are affixed to or protrude from the inner wall such that the mechanism and the baffles are not in contact with one another. For instance, where a device or devices for mixing the reaction components is located at the bottom center of the vessel, the baffles may be installed above the highest point of said means. Where multiple mechanical mechanisms are utilized, the baffles are typically configured to avoid those mechanisms. For instance, where the mechanism includes one or more sets of rotating blades, the baffle(s) may be positioned above, below, between or alongside the blades. The baffle design will ensure adequate clearance from the mechanical mechanisms. The baffle assembly is typically fixably attached to the vessel through attachment arm or arms 7 by a welding or other process that results in a substantially seamless joint. As described above, use of the attachment arms advantageously provides for efficient cleaning and/or sanitization of the baffles in that very little to no residue remains at the joint between the interior surface of the reaction vessel and the baffle following the attachment process (e.g., welding). Similarly, the baffle may be incorporated into, attached or affixed to a reaction vessel by any suitable method provided that method provides a substantially seamless attachment point (e.g., a seamless joint or boundary between materials) to provide a surface that may be simply and efficiently sanitized. A “substantially seamless attachment point”, “seamless joint”, or “crevice-free joint” typically indicates that the boundary between the baffle and the reaction vessel is substantially undetectable by either visual and/or other means (e.g., microscopy). It may also indicate that the boundary does not retain any residue from prior reactions following a standard cleaning procedure typically used by the skilled artisan to “sanitize” such equipment. The system is therefore suitable for sanitization using industry-accepted “clean-in-place” and “sterilize-in-place” systems using any suitable cleaning agent including but not limited to detergents, brushes, and/or steam. Such a boundary affords itself to simple and efficient sanitization. In some preferred embodiments, the vessels of this disclosure can include any suitable number of baffles, preferably one to ten, more preferably four to eight, most preferably eight. In some preferred embodiments, the vessels do not include any baffles.

In preferred embodiments, the optimal heat transfer surface area utilized with a particular system can be determined based on an estimation of cell culture metabolic loading based on Oxygen Uptake Rate (OUR) as well as any mechanical contribution provided by the agitator (e.g., at a power per volume of 2 HP/kGal). In preferred embodiments, the heat transfer surface area is provided on the sidewall of the bioreactor vessel to at least the maximum working volume and to the working head. In preferred embodiments, the heat transfer fluid moves through parallel flow paths along the sidewall and through the different heat transfer zones. In some embodiments, a supplemental heat exchange system could be applied to harvest lines if, e.g., a lower temperature is required during harvesting of cells from the reaction mixture.

The heat transfer systems described herein may be constructed of any material through which heat transfer fluid (e.g., gas and/or preferably liquid such as cool water (e.g., 10-12° C. depending on the application)) may be transported such that heat may be conducted to and/or absorbed from another part of the system by radiative, convective, conductive or direct contact (e.g., from the heat transfer system into the internal reaction chamber). Suitable heat transfer media include and are not limited to fluids and gases. Suitable fluids and gases include and are not limited to steam (top to bottom), hot and cold water, glycol, heat transfer oils, refrigerants, or other pumpable fluid having a desired operational temperature range. It is also possible to use multiple types of heat transfer media such that, for instance, one type of media is directed to one area of the reaction vessel and another type of media is directed to a different area of the reaction vessel (e.g., as in the zonal system described above). Mixtures of heat transfer media (e.g., 30% glycol) may also be desirable.

In preferred embodiments, the reaction mixture comprises cells, preferably non-bacterial cells, and more preferably mammalian, fish, avian, and/or insect cells at a density of about 20 to about 100 million cells per milliliter, preferably at least about 35 to 50 million cells per milliliter. For instance, in some embodiments, the bioreactor vessels of this disclosure are configured to support the parameters shown in Tables 1A, 1B (preferred embodiment) and/or Tables 2A and 2B (preferred embodiment):

TABLE 1A

| Parameter | Value | Units |
|---|---|---|
| Target Cell Density | 20-100 | 10³-10⁶ |
| | | Cells/mL |
| pH | 6-9 | Units |
| Temperature | 20-60 | ° C. |
| Dissolved Oxygen | 20-60 | % of Sat |

TABLE 1B
(preferred embodiment)

| Parameter | Value | Units |
|---|---|---|
| Target Cell Density | 35 | 10⁶ Cells/mL |
| pH | 6.8-7.8 | Units |
| Temperature | 33-40 | ° C. |
| Dissolved Oxygen | 30-50 | % of Sat |

TABLE 2A

| Parameter | Value | Units |
|---|---|---|
| Target Cell Density | 30-70 | 10⁵-10⁷ |
| | | Cells/mL |
| Target Oxygen Transfer Rate | 15-30 | mmol/L/h |
| (OTR) | | |
| Agitator Power | 0.1-5 | HP/kGal |
| Dissolved Oxygen | 20-50 | % of Sat |
| Temperature | 33-40 | ° C. |
| pH | 6-9 | Units |

TABLE 2B
(preferred embodiment)

| Parameter | Value | Units |
|---|---|---|
| Target Cell Density | 50 | 10⁶ Cells/mL |
| Target Oxygen Transfer Rate | 20 | mmol/L/h |
| (OTR) | | |
| Agitator Power | 1 | HP/kGal |
| Dissolved Oxygen | 30 | % of Sat |
| Temperature | 37 | ° C. |
| pH | 7.3 | Units |

Typical bioreactors have an aspect ratio, defined herein as the liquid depth (LD) to bioreactor vessel diameter (D) ratio, of about 1.0-1.5, preferably 1.25-1.5, or for larger bioreactors up to about 2. The bioreactors disclosed herein have an aspect ratio of about 1.5 to about 3.0, with a preferred aspect ratio of about 2.4 (in some preferred embodiments, about 2.36). These aspect ratios, especially of about 2.36 and higher, support processes exhibiting the parameters described in Tables 1 and 2 (e.g., about 35 million cells/mL, about 2,250,000 L reactor capacity). The systems disclosed herein are also configured to be manufacturable at one site (e.g., and shipped if necessary), adjustable depending on the particular cells being grown, maintain sterility, and operate with commercially available filters and the like. In some preferred embodiments, the bioreactors of this disclosure can comprise the dimensions and parameters shown in Table 3:

TABLE 3

| | | | |
|---|---|---|---|
| Production Reactor | 125 | 250 | 315 |
| Working Volume, m³ | | | |
| Production Reactor | 157 | 313 | 394 |
| Total Volume, m³ | | | |
| Head/s Type | e.g., ASME | e.g., ASME | e.g., ASME |
| | F&D, ASME | F&D, ASME | F&D, ASME |
| | 80/10 | 80/10 | 80/10 |
| | (preferred) | (preferred) | (preferred) |
| Inner Diameter, in | 162 | 204 | 220 |
| Unaerated Liquid | 382 | 482 | 521 |
| Height, in | | | |
| L/D Liquid | 2.4 | 2.4 | 2.4 |

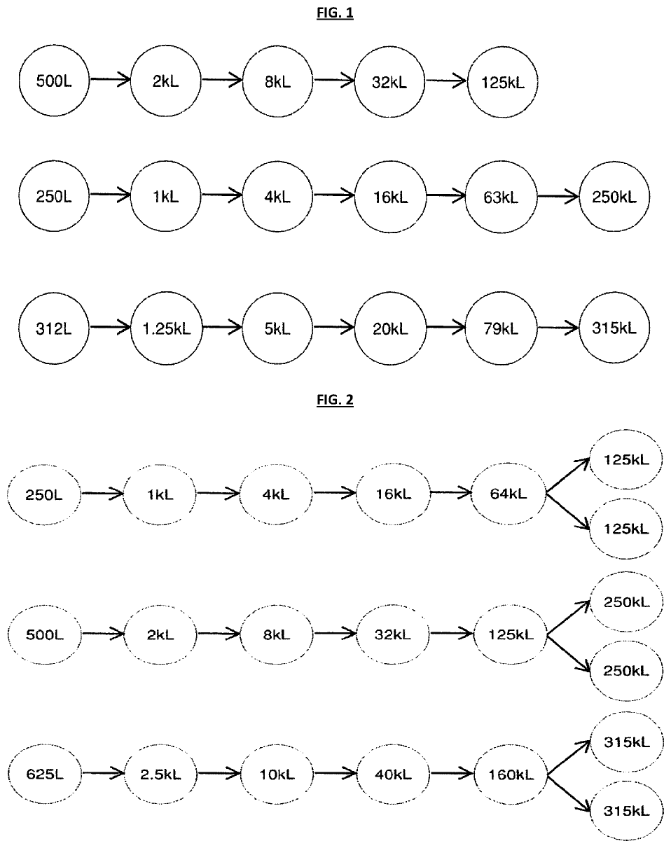

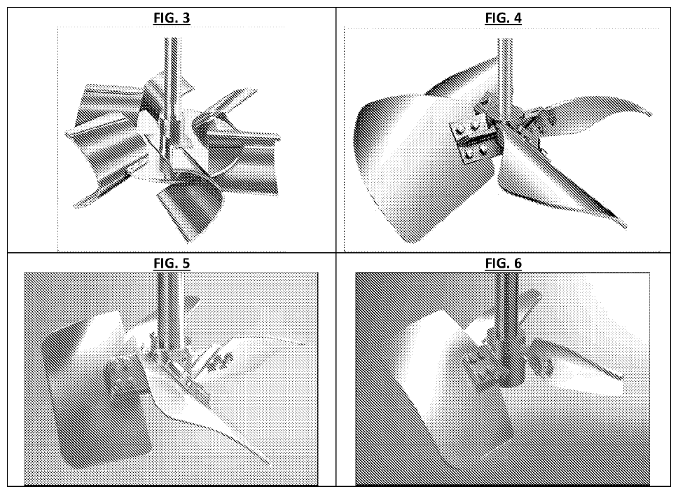

In some embodiments, a “seed train” technique is used to provide an initial volume of cells into the bioreactor. This “seed train” is typically made up of a number of smaller bioreactors that allow the cell volume to sufficiently expand to desired densities as the contents of the bioreactors are transferred to subsequent bioreactors. All or portions of a bioreactor contents can be transferred into subsequent bioreactors. For instance, an exemplary bioreactor train is shown in each of FIGS. 1 and 2).

The bioreactor vessels disclosed herein are typically, but not necessarily, constructed of metal and usually, but not necessarily, from a corrosion-resistant alloy. For instance, suitable materials may include, without limitation, sheet/plate stock (and/or dimple-jacket material for, e.g., heat transfer systems). Suitable exemplary materials include, for example, carbon steel, stainless steel (e.g., 304, 304L, 316, 316L, 317, 317L, AL6XN), aluminum, Inconel® (e.g., Inconel 625, Chronin 625, Altemp 625, Haynes 625, Nickelvac 625 and Nicrofer 6020), Incoloy®, Hastelloy (e.g., A, B, B2, B3, B142T, Hybrid-BC1, C, C4, C22, C22HS, C2000, C263, C276, D, G, G2, G3, G30, G50, H9M, N, R235, S, W, X), and Monel®, titanium, Carpenter 20®, among others. It is understood, however, that other materials besides or in addition to a corrosion-resistant alloy such as, but without limitation, plastic, rubber, and mixtures of such materials may also be suitable. A “mixture” of materials may refer to either an actual mixture per se to form a combined material or the use of various materials within the system (e.g., an alloy reactor shell and rubber baffle components).

The reaction mixture typically includes a liquid cell culture media suitable for maintaining the viability and growth of the cells of interest. As mentioned above, the bioreactor vessels disclosed herein can accommodate the growth of various types of cells including but not limited to mammalian, fish, avian, and/or insect cells. Exemplary cell culture medias would be any of those typically used for culturing such cells and modified as needed to allow for viability and growth within the bioreactor vessel (e.g., to the densities disclosed herein). Typically, the cell culture media and any other liquids introduced into the bioreactor vessel during the cell growth/expansion process is sterile. The cell expansion process could take different forms such as batch (i.e., in which the entire volume of media is introduced at a single time), fed-batch (in which media and nutrients are added throughout the growth time) or in a process intensification form (i.e., in which an external device such as a filter is used to exchange media/nutrients to allow higher cell densities). Other processes may also be suitable as would be understood by those of ordinary skill in the art. Following the reaction (e.g., growth to 50 million cells/ml), cell harvest is typically performed directed from the bioreactor vessel to minimize the risk of contamination. In some embodiments, following the reaction (e.g., growth to 50 million cells/ml), cell harvest can be performed directly from the bioreactor vessel into a sterile transfer line(s) to minimize the risk of contamination. In some embodiments, the entire contents of the bioreactor vessel could be harvested simultaneously, or in some embodiments only a portion could be harvested, and then additional media introduced into the system to continue cell expansion in a manner known as draw and fill.

In some embodiments, the system could comprise piping (e.g., tubing) that can be independently cleaned and sterilized from other sections of the system to allow the bioreactor to sterilely accept liquid additions into the product stream. Exemplary liquid additions can be, for instance, cell culture media or its individual components, cell culture fluid for inoculation, basic or acidic solutions to control pH, glucose or another sugar for cell growth, antifoam, and/or the like. In some embodiments, each of these liquid additions could be fed from a previous bioreactor as in a train of bioreactors, other holding vessel(s) of a proper size for reaction, and/or a header system that could supply multiple of these bioreactors.

Cleaning of the internal reaction chamber (i.e., production reactor) could be achieved through a clean-in-place (CIP) skid providing relevant acid and caustic washes and clean water rinses through sprayballs and spraywands within the vessel. Table 4 shows total flowrates based on three different types of sprayballs, static, single axis dynamic and multi axis dynamic. In preferred embodiments, flowrates are based on ASME BPE flow rate guidelines. An “empty” sterilization, utilizing clean steam (i.e., steam prepared from a purified water source) or culinary grade stream as the same is known in the field, can also be used to clean the internal reaction chamber (i.e., the production reactor). Time and temperature can be adjusted to align with desired sterilization requirements. An exemplary suitable sterilization can include heating the empty vessel and its sterile boundary up to 125° C. and hold this temperature for 30 minutes. Steam would be replaced with clean air and the vessel would cool down to allow for media addition. Time and temperature can be adjusted to align with the desired sterilization requirements of the user.

TABLE 4

| Production | | Maximum Flow - | Maximum Flow - |
|---|---|---|---|
| Reactor | Maximum Flow - | Single Axis | Multi Axis |
| Working | Static Sprayballs, | Dynamic | Dynamic |
| Volume, m³ | GPM | Sprayballs, GPM | Sprayballs, GPM |
| 125 | 127 | 97 | 64 |
| 250 | 160 | 122 | 80 |
| 315 | 173 | 132 | 86 |

Sufficient mixing and gas dispersion are key to maintaining optimal cell viability and growth in a bioreactor vessel (i.e., within the interior reaction chamber). Such mixing and dispersion is typically accomplished using an agitation system comprising an agitator including one or more impellers, preferably low shear impellers, in some preferred embodiments hydrofoil impellers, or in some preferred embodiments Rushton impellers. Table 5 provides exemplary agitator sizing for the bioreactor vessels disclosed herein. While particular impellers are listed in Table 10, it should be understood that any suitable impeller(s) can be used. In preferred embodiments, the agitator would include two (2) to about six (6) impellers, preferably four (4) impellers, to provide sufficient mixing throughout the reaction mixture. In addition, the number of heat transfer baffles can be from zero to eight (or more if appropriate), with four baffles being a preferred embodiment.

TABLE 5

| | | | |
|---|---|---|---|
| Production Reactor | 125 | 250 | 315 |
| Working Volume, m³ | | | |
| Type of Impeller | ABEC Low | ABEC Low | ABEC Low |
| | Shear | Shear | Shear |
| Vessel ID, in | 162 | 204 | 220 |
| Orientation | Center | Center | Center |
| Pumping Direction | Down | Down | Down |
| Number of Baffles | 4 | 4 | 4 |
| Number of Impellers | 4 | 4 | 4 |

From these exemplary designs and a preferred range of about 5 to about 40 mmol/L/hr (in some preferred embodiments about 20 mmol/l/h (see Tables 6-7)) OTR and about 0.5 to about 3.0 sHP/kGal (in some preferred embodiments about 1 sHP/kGal (see Tables 6-7)) baseline (see Tables 2A and 2B (20 mmol/l/h and 1 sHP/kGal)), gas flows can be estimated. Gassing rates using both air and air with oxygen supplementation can be utilized. Tables 6 and 7 show the outputs for these exemplary designs. For embodiments in which gas flow includes supplemental oxygen (Table 6), total flows are on the order of magnitude of 0.1 vvm. Air-only flows (Table 7) are known to rise correspondingly based on the mol fraction of oxygen being delivered. Given the higher aspect ratio, an agitator designed around hydrofoil impellers is preferred in some embodiments. The low shear impellers included in the bioreactor vessel agitators used to produce the date in Table 7 were replaced by hydrofoil type impellers and mass transfer correlations were derived therefrom as shown in Tables 8 (supplemental oxygen) and 9 (air only).

TABLE 6

| | Oxygen Transfer | | |
|---|---|---|---|
| Production Reactor | Rate, OTR - | Air Flow - | Oxygen Flow - |
| Working Volume, m³ | mmol O₂/L-hr | SLPM | SLPM |
| 125 | 20.6 | 12,500 | 875 |
| 250 | 20.6 | 25,000 | 1,750 |
| 315 | 20.6 | 30,000 | 2,000 |

TABLE 7

| | Oxygen Transfer | | |
|---|---|---|---|
| Production Reactor | Rate, OTR - | Air Flow - | Oxygen Flow - |
| Working Volume, m³ | mmol O₂/L-hr | SLPM | SLPM |
| 125 | 20.6 | 20,500 | 0 |
| 250 | 20.6 | 41,000 | 0 |
| 315 | 20.6 | 49,500 | 0 |

TABLE 8

| | Oxygen Transfer | | |
|---|---|---|---|
| Production Reactor | Rate, OTR - | Air Flow - | Oxygen Flow - |
| Working Volume, m³ | mmol O₂/L-hr | SLPM | SLPM |
| 125 | 20.6 | 5,000 | 500 |
| 250 | 20.6 | 10,000 | 1,000 |
| 315 | 20.6 | 13,100 | 1,100 |

TABLE 9

| | Oxygen Transfer | | |
|---|---|---|---|
| Production Reactor | Rate, OTR - | Air Flow - | Oxygen Flow - |
| Working Volume, m³ | mmol O₂/L-hr | SLPM | SLPM |
| 125 | 20.6 | 8,125 | 0 |
| 250 | 20.6 | 16,250 | 0 |
| 315 | 20.6 | 19,750 | 0 |

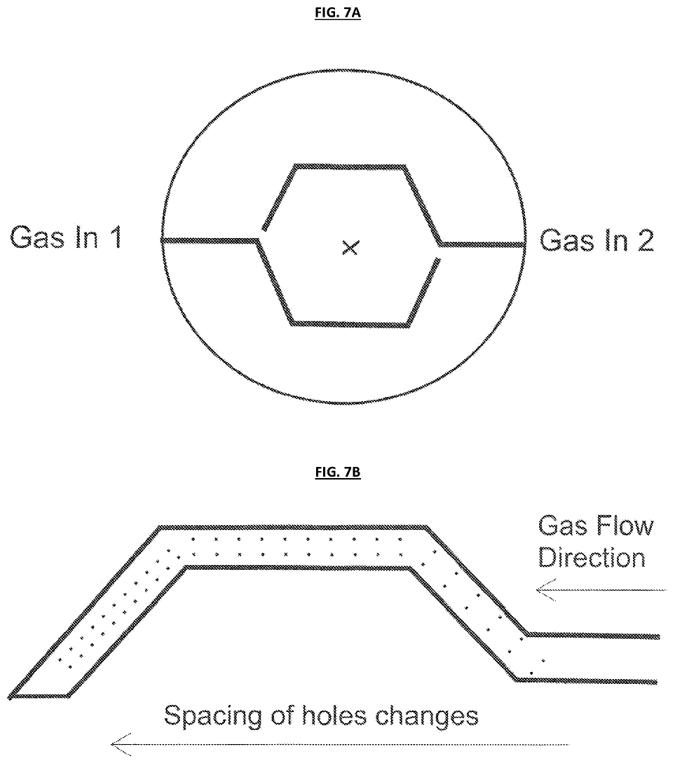

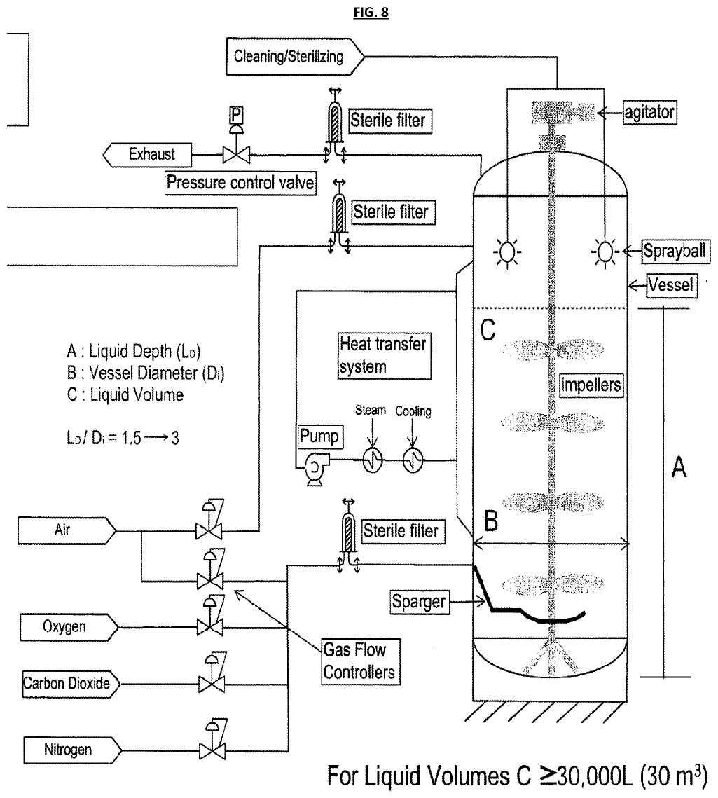

Suitable impellers can be any available to those of ordinary skill in the art. Exemplary hydrofoil impeller designs that could be used with the bioreactor vessels disclosed herein are shown in FIGS. 3-6. The characteristics of the impellers shown in FIGS. 3-4 are shown in Table 10 while those of the impellers shown in FIGS. 5-6 are shown in Table 11.

TABLE 10

| | | | |
|---|---|---|---|
| Production Reactor Working | 125 | 250 | 315 |
| Volume, m³ | | | |
| Vessel ID, in | 162 | 204 | 220 |
| Upper Impeller Type | Wide Blade | Wide Blade | Wide Blade |
| | Hydrofoil | Hydrofoil | Hydrofoil |
| Mid Impeller Quantity | 3 | 3 | 3 |
| Mid Impellers Type | Wide Blade | Wide Blade | Wide Blade |
| | Hydrofoil | Hydrofoil | Hydrofoil |
| Lower Impeller Type | Rushton | Rushton | Rushton |
| Number of Baffles | 4 | 4 | 4 |
| Pumping Direction | Down | Down | Down |
| Total Number of Impellers | 5 | 5 | 5 |

TABLE 11

| | | | |
|---|---|---|---|
| Production Reactor Working | 125 | 250 | 315 |
| Volume, m³ | | | |
| Vessel ID, in | 162 | 204 | 220 |
| Upper Impeller Type | Wide Blade | Wide Blade | Wide Blade |
| | Hydrofoil | Hydrofoil | Hydrofoil |
| Mid Impeller Quantity | 2 | 2 | 2 |
| Mid Impellers Type | Wide Blade | Wide Blade | Wide Blade |
| | Hydrofoil | Hydrofoil | Hydrofoil |
| Lower Impeller Type | Wide Blade | Wide Blade | Wide Blade |
| | Hydrofoil | Hydrofoil | Hydrofoil |
| Number of Baffles | 4 | 4 | 4 |
| Pumping Direction | Down | Down | Down |
| Total Number of Impellers | 4 | 4 | 4 |

Manufacturability is another key determination of the feasibility of an agitator at these scales. Given the size of the exemplary impellers shown in FIGS. 3-6, one piece construction as may be desired in a sterile environment could pose a manufacturing challenge (e.g., in-tank couplings for shaft handling and manufacturing would be required). In some embodiments, individual impeller blades can be bolted onto a welded hub for handling purposes. It is preferred, but not necessary, that all connections to be of a sanitary design as outlined in ASME BPE, Appendix 10.3. Steady bearings can also be used to handle shaft deflection and could be used at the bottom of the bioreactor vessel (e.g., in embodiments in which the agitator gear box is mounted at the top of the bioreactor vessel. In some embodiments, the agitator can be sealed to the bioreactor vessel by a cartridge type mechanical seal. In some embodiments, a pressurized, lubricated seal utilizing clean steam condensate as the lubricant can be utilized. In some embodiments, a single dry running seal with a sanitary gland.

In some embodiments, the reactor system of this disclosure can comprise at least two spargers, each comprising a fluidic channel and at least one section comprising multiple perforations through which the at least one component is introduced into the reaction mixture through the bottom section of the internal reaction chamber, optionally wherein the sections comprising multiple perforations together provide an circular, oval, hexagonal, square, rectangular or other shaped structure (see a preferred embodiment shown in FIGS. 7A and 7B showing the orientation of the sparger positioned at the bottom of the bioreactor vessel and the spacing of holes represented as dots through which gas traverses, respectively). In some preferred embodiments, a single sparger may be included (e.g., having a circular, oval, hexagonal, square, rectangular or other shape). In preferred embodiments, the spacing of the perforations in the sparger(s) becomes less (e.g., the perforations are more numerous and closer to one another) as the sparger structure extends away from the end the source of the gas entering the sparger.

In some embodiments, the fluidic channels used as supply lines to the bioreactor vessel (e.g., the interior reaction chamber thereof) can comply with typical carbon or stainless steel piping specifications and would preferably be sanitary (especially those having direct product contact, connecting other vessels providing sterile liquids to the bioreactor vessel, gas supply, gas exhaust, CIP distribution and clean steam distribution). Other utilities such as plant steam and chilled water are not considered to be of a sanitary design. In preferred embodiments, the line sizing for sanitary lines allows the use of ASME BPE tubing, which is the standard for sanitary applications in biopharma. Diaphragm valves can be used within the sterile boundary of the bioreactor vessel. Other valve types such as sanitary butterfly, ball, and mix-proof valves could be used in the construction within the sterile boundary and to direct other fluids where a sanitary construction would be desired. In preferred embodiments, the material for these lines would follow typical biopharma applications and be 316L stainless steel. The surface finishes of the bioreactor vessel, and particularly of the interior reaction chamber, preferably meet a minimum 30 pinch Ra mechanical polish. A suitable surface could have a lesser Ra (i.e., <30 pinch) if it were combined with a technique such as electropolishing. Any elastomers in the sanitary lines are preferably USP/FDA compliant (e.g., EPDM and/or Platinum Cured Silicone). In preferred embodiments, the bioreactor vessels can have a shell of 316L stainless steel with a mechanical polish that would meet a minimum 30 pinch Ra (unless combined with, e.g., electropolishing), jacket material can be 304L stainless steel and insulated using insulation sheathing material. In some embodiments, an alternative duplex steel (e.g., UNS S32205) could be used to construct the bioreactor vessel. Depending on the final pressure and load ratings of the vessels, such an alternative could decrease cost due to decreased thicknesses. In preferred embodiments, ports for analytical probes such as Dissolved Oxygen, pH and pCO₂ along the sidewall of the bioreactor vessel can be included.

In preferred embodiments, the systems described herein may also include one or more manual and/or automated control systems (i.e., not requiring continuous direct human intervention, or constant direct human intervention), including but not limited to one or more remotely controlled control systems. For instance, a control system may continuously monitor one or more conditions occurring within any of the components of the system, preferably between at least any two components of the system. Such control systems typically comprise one or more general purpose computers including software for processing such information and manually or automatically adjusting the desired parameters of the reaction as required by a particular process. Thus, in some preferred embodiments, the control system is automated (e.g., using software). In some preferred embodiments, the systems described herein can include one or more automation system(s) for control and monitoring of process conditions and process sequencing. In some preferred embodiments, the automation system includes hardware (automation system hardware) including but not limited to commercially-available Programmable Logic Controllers (PLC), Distributed Control Systems (DCS), and/or one or more Human-Machine Interfaces (HMI). In preferred embodiments, the automation system hardware is programmed for control and monitoring of process conditions and process sequencing. Process control and monitoring parameters that can be controlled by such manual and/or preferably automated systems include but are not limited to dissolved oxygen, pCO₂, temperature, liquid level, foam detection/control, gassing/mass flow, headspace pressure, pH, agitator speed, viable cell density, exhaust gas analysis and spectroscopy methods including Ultraviolet (UV) and Raman; and can incorporate specific control algorithms such as exponential feeding. Large bioreactor process sequences that can be controlled can include clean-in-place (CIP), sterilization-in-place (SIP), pressure hold testing, vessel charging, cell growth, reagent addition and/or cell harvest processes. Process control and monitoring can also include integration/interfacing of external process systems supplying or servicing the large bioreactor, including reagent addition tanks, CIP systems, SIP systems, liquid sterilization systems and harvest systems. Process control, monitoring and sequencing data may be collected and stored as a batch record. Exemplary automatically-controlled systems are shown in FIGS. 10-12. Such systems preferably include Ethernet-based Redundant Plant Control Network connection to two redundant network switches (not shown) in each remote I/O panel and redundant ethernet connection from switches to HMI and to Ethernet I/O (shown) inside remote I/O panel (FIGS. 10-12, A, B (ethernet)).

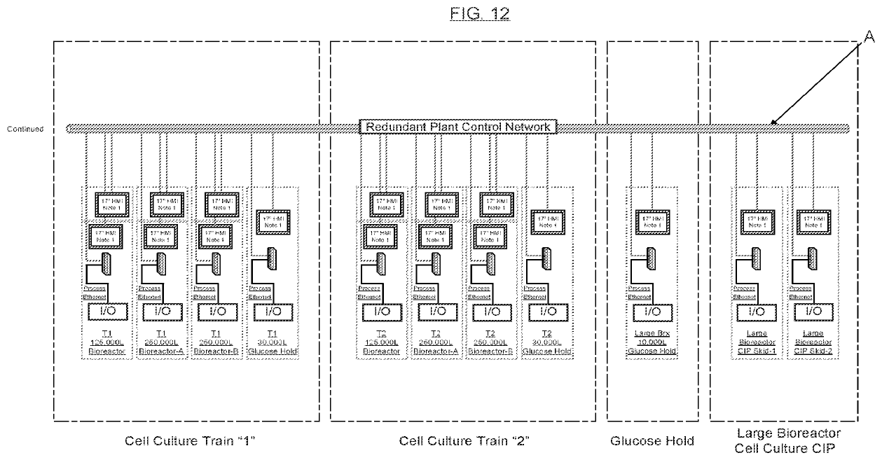

Exemplary bioreactor systems including automation systems are shown in FIGS. 10-12. As shown in FIG. 10, certain preferred embodiments of bioreactor systems and/or subsystems of bioreactor systems can include a media preparation (“Media Prep”) subsystem is connected to a nutrient preparation subsystem (“Nutrient Prep”), and a large media preparation subsystem (“Large Media Prep”), that can include vessels of the same or different sizes, clean-in-place skids, connected by a Redundant Plant Control Network, along with the various subsystems thereof. As shown in FIG. 11, certain preferred embodiments of bioreactor systems and/or subsystems of bioreactor systems can include a large media preparation subsystem (“Large Media Prep”), that can include vessels of the same or different sizes, one or more clean-in-place skids, connected by a Redundant Plant Control Network, along with the various subsystems thereof. As shown in FIGS. 12, certain preferred embodiments of bioreactor systems and/or subsystems of bioreactor systems can include one or more cell culture trains and/or a nutrient vessel (e.g., Glucose Hold), and/or a Large Bioreactor Cell CIP system (FIG. 12), that can include vessels of the same or different sizes, one or more clean-in-place skids, connected by a Redundant Plant Control Network, along with the various subsystems thereof. Preferred embodiments of bioreactor systems including supply lines, returns and the like. In some embodiments, certain preferred bioreactor systems can include multiple bioreactor vessels fluidly connected in series of, for instance, 25,000; 32,000; 40,000; 50,000; 125,000; and/or 250,000 L. In certain preferred embodiments, the various vessels, subsystems, and/or bioreactor systems at least two trains (e.g., two, three, four, or five) of vessels fluidly connected in series, wherein the vessels are 500 L, 2,000 L, 8,000 L, 32,000 L, 125,000 L, and 250,000 L (in preferred embodiments each train includes at least one 125,000 L vessels feeding into two 250,000 L vessels), that can also be fluidly connected to glucose hold vessels (e.g., 30,000 L). Other embodiments of control systems can also be used, as would be understood by those of ordinary skill in the art.

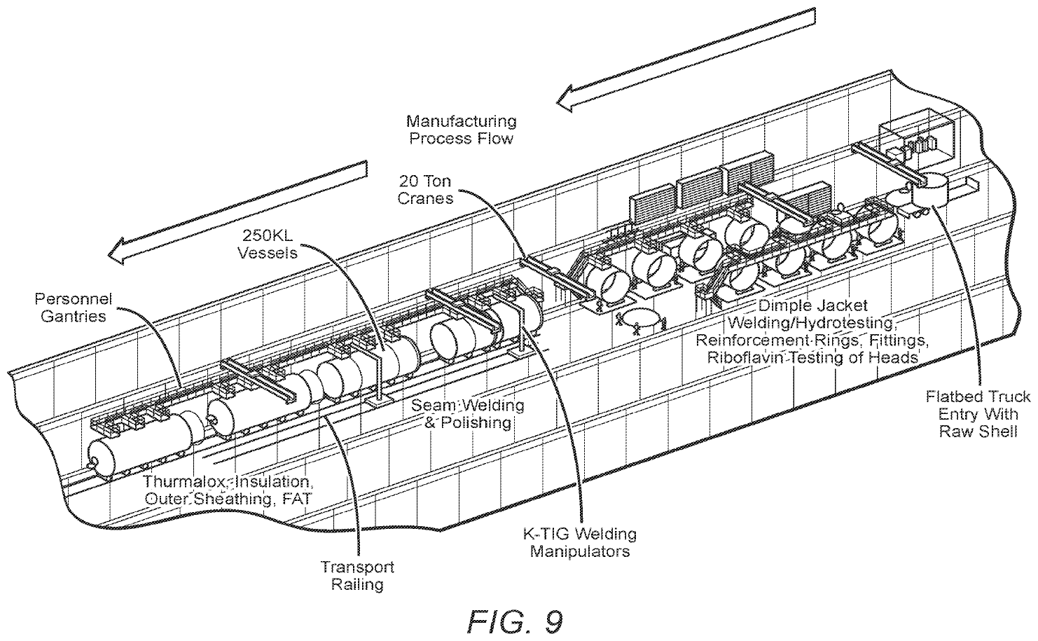

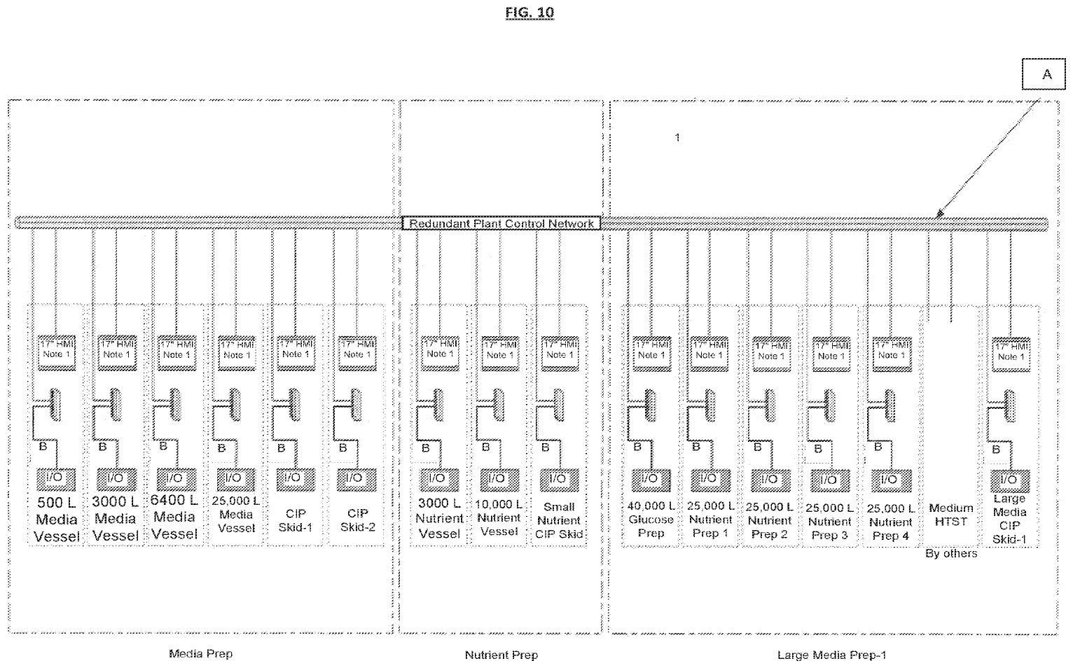

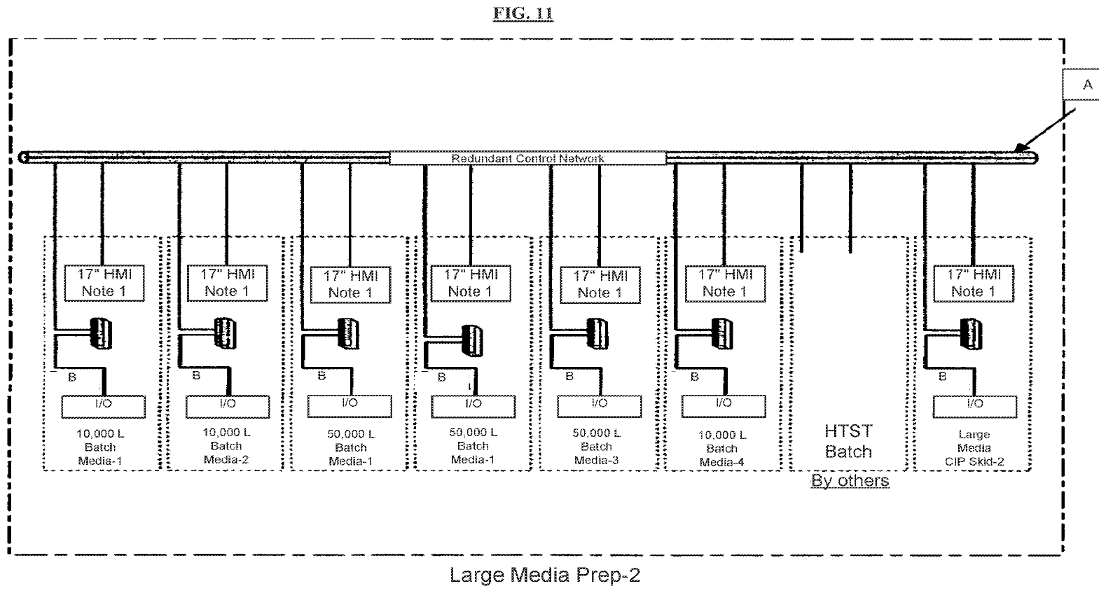

Thus, this disclosure provides the following preferred aspects and preferred embodiments:
1. A bioreactor system comprising:
a) a vessel comprising internal reaction chamber:
   a. configured to contain at least about 30,000 liters (L), at least about 50,000 L, at least about 75,000 L, at least about 100,000 L, at least about 125,000 liters L, at least about 250,000 L, or at least about 315,000 L of a reaction mixture comprising cells, liquid, and/or gas;
   b. a liquid depth (LD) to bioreactor vessel diameter (D) ratio of 1.5-3, the ratio optionally being about 2.4 (e.g., in some preferred embodiments about 2.36); and,
   c. top and bottom sections, wherein at least the bottom section is in contact with the reaction mixture;
b) at least one heat transfer system at least partially surrounding at least one area of the internal reaction chamber and being configured to maintain the reaction mixture in said area at a pre-selected temperature;
c) at least one fluidic channel (sparger) providing at least one component of the reaction mixture, said at least one component being selected from the group consisting of air, oxygen, carbon dioxide (CO₂), and/or nitrogen through the bottom section;
d) at least one fluidic channel providing air to the top section of the internal reaction chamber;
e) at least one agitator for mixing said reaction mixture, the agitator comprising multiple low shear impellers, said low shear impellers optionally being hydrofoil or Rushton impellers;
f) at least one fluidic channel for removing exhaust from the top section of the internal reaction chamber; and,
g) at least one cleaning and/or sterilizing system for cleaning and/or sterilizing the internal reaction chamber, the at least one cleaning and/or sterilizing system being fluidly connected to the top section of the internal reaction chamber.
2. The bioreactor system of aspect 1 comprising at least two spargers, each comprising a fluidic channel and at least one section comprising multiple perforations through which the at least one component is introduced into the reaction mixture through the bottom section of the internal reaction chamber, optionally wherein the sections comprising multiple perforations together provide an circular, oval, hexagonal, square, rectangular or other shaped structure (see a preferred embodiment shown in FIGS. 7A and 7B showing the orientation of the sparger positioned at the bottom of the bioreactor vessel and the spacing of holes represented as dots through which gas traverses, respectively). In some preferred embodiments, a single sparger may be included (e.g., having a circular, oval, hexagonal, square, rectangular or other shape). In preferred embodiments, the spacing of the perforations in the sparger(s) becomes less (e.g., the perforations are more numerous and closer to one another) as the sparger structure extends away from the end the source of the gas entering the sparger.
3. The bioreactor system of any preceding aspect comprising a single agitator comprising multiple impellers, optionally four impellers, further optionally wherein said impellers are hydrofoil or Rushton impellers.
4. The bioreactor system of any preceding aspect wherein the internal reaction chamber is configured to contain at least about 125,000 liters L, at least about 250,000 L, or at least about 315,000 L of a reaction mixture comprising cells, liquid, and gas.
5. The bioreactor system of any preceding aspect wherein the reaction mixture comprises cells at a density of about 20 to about 100 million cells per milliliter, optionally about 50 million cells per milliliter.
6. The bioreactor system of any preceding aspect wherein the at least one fluidic channel (sparger) provides oxygen into the reaction mixture at a transfer rate of at least about 20 mmol/L/hour.
7. The bioreactor system of any preceding aspect wherein the heat transfer system comprises heat transfer fluid having temperature of at least about 10-12° C. (higher during, e.g., the control phase), optionally wherein said heat transfer fluid is water.
8. The bioreactor system of any preceding aspect wherein the heat transfer system comprises a dimpled jacket.
9. The bioreactor system of any preceding aspect wherein the at least one cleaning and/or sterilizing system applies a cleaning solution, optionally an acid, to the interior of the internal reaction chamber, further optionally wherein the cleaning system comprises at least one sprayball and/or spraywand.
10. The bioreactor system of any preceding aspect wherein the reaction mixture is produced from a series of seed trains through which the volume of the reaction mixture is incrementally increased, optionally beginning at a volume of about at least 250 L. In some embodiments, however, the series of seed trains could begin at much lower levels, such as the vial level (e.g., 25 ml).
11. The bioreactor system of any preceding aspect wherein the reaction mixture is maintained by perfusion.
12. The bioreactor system of any preceding aspect wherein foaming, if present, of the reaction mixture is controlled using a chemical anti-foam agent and/or a mechanical anti-foam system.
13. The bioreactor system of any preceding aspect wherein the liquid in the reaction mixture comprises cell culture media.
14. The exemplary bioreactor system comprising the components illustrated in FIG. 8. This exemplary bioreactor system comprises the vessel comprising the internal reaction chamber configured to contain at least about 30,000 liters (L), at least about 50,000 L, at least about 75,000 L, at least about 100,000 L, at least about 125,000 liters L, at least about 250,000 L, or at least about 315,000 L of a reaction mixture comprising cells, liquid, and/or gas, multiple impellers connected to a shaft which is in turn connected to an agitator motor, at least one sparger fluidly connected to a fluidic channel connected to a sterile filter and fluidic channels through which air, oxygen, carbon dioxide, and/or nitrogen flow into the sparger (along with sources/vessels providing the same), and gas flow controllers; at least one heat transfer system comprising a jacket through which heat transfer fluid flows to cool the reaction mixture (“liquid volume”), at least one pump, at least one source of heat transfer fluid and/or a source of heat (e.g., “steam”) or cooling connected to a fluidic channel through which the heat transfer fluid flows; a section of the vessel above the reaction mixture (liquid volume) (e.g., a headspace) that can comprise at least one source of air and a fluidic channel through which the same is introduced into the headspace, at least one sprayball(s) or other structure through which cleaning fluid, steam, or other gas (and a source thereof) can flow into the empty vessel for cleaning the vessel once the reaction has ended and the vessel emptied; and an exhaust system comprising at least one sterile filter, at least one pressure control valve, where the exhaust system exhausts gas (e.g., humid gas) into the environment.
15. A method for manufacturing a bioreactor system of any preceding aspect, the method comprising:
   a. modifying a structural shell comprising at least one section of the vessel with a heat transfer system that is optionally a dimple jacket; reinforcement rings; and/or fittings; to produce a modified structural shell;
   b. seam welding multiple modified structural shells to connect the same to one another, thereby producing seams at the interface between the modified structural shells, and polishing said seams;
   c. insulating, coating, painting, and/or installing an outer sheathing to the connected modified structural shells connected in step b); and,
   d. transporting the products of steps a), b) and/or c) using at least one crane and/or track or railing (see, e.g., FIG. 9).
16. A bioreactor system and/or method of any preceding aspect wherein the bioreactor system comprises and/or is operably connected to an automated control system.

Other embodiments, aspects, advantages of the systems and methods of using the same are also provided herein, as would be understood by those of ordinary skill in the art.

The terms “about”, “approximately”, and the like, when preceding a list of numerical values or range, refer to each individual value in the list or range independently as if each individual value in the list or range was immediately preceded by that term. The terms mean that the values to which the same refer are exactly, close to, or similar thereto. Optional or optionally means that the subsequently described event or circumstance can or cannot occur, and that the description includes instances where the event or circumstance occurs and instances where it does not. Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed.

All references cited within this disclosure are hereby incorporated by reference in their entirety. Certain embodiments are further described in the following examples. These embodiments are provided as examples only and are not intended to limit the scope of the claims in any way. While certain embodiments have been described in terms of the preferred embodiments, it is understood that variations and modifications will occur to those skilled in the art. Therefore, it is intended that the appended claims cover all such equivalent variations that come within the scope of the following claims.

1. A bioreactor system comprising:
a) a vessel comprising internal reaction chamber:
configured to contain at least about 30,000 liters (L), at least about 50,000 L, at least about 75,000 L, at least about 100,000 L, at least about 125,000 liters L, at least about 250,000 L, or at least about 315,000 L of a reaction mixture comprising cells, liquid, and/or gas;
a liquid depth (LD) to bioreactor vessel diameter (D) ratio of 1.5-3, the ratio optionally being about 2.4, optionally about 2.36; and,
top and bottom sections, wherein at least the bottom section is in contact with the reaction mixture;
b) at least one heat transfer system at least partially surrounding at least one area of the internal reaction chamber and being configured to maintain the reaction mixture in said area at a pre-selected temperature;
c) at least one fluidic channel (sparger) providing at least one component of the reaction mixture, said at least one component being selected from the group consisting of air, oxygen, carbon dioxide (CO₂), and/or nitrogen through the bottom section;
d) at least one fluidic channel providing air to the top section of the internal reaction chamber;
e) at least one agitator for mixing said reaction mixture, the agitator comprising multiple low shear impellers, said low shear impellers optionally being hydrofoil or rushton impellers;
f) at least one fluidic channel for removing exhaust from the top section of the internal reaction chamber; and,
g) at least one cleaning and/or sterilizing system for cleaning and/or sterilizing the internal reaction chamber, the at least one cleaning and/or sterilizing system being fluidly connected to the top section of the internal reaction chamber.

2. The bioreactor system of claim 1 comprising at least two spargers, each comprising a fluidic channel and at least one section comprising multiple perforations through which the at least one component is introduced into the reaction mixture through the bottom section of the internal reaction chamber, optionally wherein the sections comprising multiple perforations together provide an essentially hexagonal structure.

3. The bioreactor system of claim 1 comprising a single agitator comprising multiple impellers, optionally four impellers, further optionally wherein said impellers are hydrofoil or rushton impellers.

4. The bioreactor system of claim 1 wherein the internal reaction chamber is configured to contain at least about 125,000 liters L, at least about 250,000 L, or at least about 315,000 L of a reaction mixture comprising cells, liquid, and gas.

5. The bioreactor system of claim 1 wherein the reaction mixture comprises cells at a density of about 20 to about 100 million cells per milliliter, optionally about 50 million cells per milliliter.

6. The bioreactor system of claim 1 wherein the at least one fluidic channel or sparger provides oxygen into the reaction mixture at a transfer rate of at least about 20 mmol/L/hour.

7. The bioreactor system of claim 1 wherein the heat transfer system comprises heat transfer fluid having temperature of at least about 10-12° C., optionally wherein said heat transfer fluid is water.

8. The bioreactor system of claim 1 wherein the heat transfer system comprises a dimpled jacket.

9. The bioreactor system of claim 1 wherein the at least one cleaning and/or sterilizing system applies a cleaning solution, optionally an acid, to the interior of the internal reaction chamber, further optionally wherein the cleaning system comprises at least one sprayball and/or spraywand.

10. The bioreactor system of claim 1 wherein the reaction mixture is produced from a series of seed trains through which the volume of the reaction mixture is incrementally increased, optionally beginning at a volume of about 25 ml to at least 250 L.

11. The bioreactor system of claim 1 wherein the reaction mixture is maintained by perfusion.

12. The bioreactor system of claim 1 wherein foaming, if present, of the reaction mixture is controlled using a chemical anti-foam agent and/or a mechanical anti-foam system.

13. The bioreactor system of claim 1 wherein the liquid in the reaction mixture comprises cell culture media.

14. A method for manufacturing a bioreactor system of claim 1, the method comprising:
a. modifying a structural shell comprising at least one section of the bioreactor vessel with a heat transfer system that is optionally a dimple jacket; reinforcement rings; and/or fittings; to produce a modified structural shell;
b. seam welding multiple modified structural shells to connect the same to one another, thereby producing seams at the interface between the modified structural shells, and polishing said seams;
c. insulating, coating, painting, and/or installing an outer sheathing to the connected modified structural shells connected in step b); and,
d. transporting the products of steps a), b) and/or c) using at least one crane and/or track or railing.

15. A bioreactor system of claim 1 wherein the bioreactor system comprises and/or is operably connected to an automated control system.

16. A method of claim 14 wherein the bioreactor system comprises and/or is operably connected to an automated control system.
